(19) United States
(12) Patent Application Publication
(10) Pub. No.: US 2014/0206940 A1
(43) Pub. Date: Jul. 24, 2014

(54) METHOD OF TISSUE SEPARATION USING FLUIDIC PULSES TO MINIMIZE TISSUE TRAUMA DURING CATHETER INSERTION

(71) Applicant: The Charles Stark Draper Laboratory, Inc., Cambridge, MA (US)

(72) Inventor: Kevin A. Hufford, St. Petersburg, FL (US)

(73) Assignee: The Charles Stark Draper Laboratory, Inc., Cambridge, MA (US)

(21) Appl. No.: 14/163,136

(22) Filed: Jan. 24, 2014

Related U.S. Application Data
(60) Provisional application No. 61/756,304, filed on Jan. 24, 2013.

(51) Int. Cl. A61F 9/00; A61B 1/06

(52) U.S. Cl. CPC A61F 9/0017; A61B 1/06
USPC 600/158; 604/521; 604/151; 604/67

(57) ABSTRACT

The method and system disclosed herein uses a pulsatile, dilatory bubble at the tip of a catheter to facilitate tissue separation and ease catheter insertion for, among other applications, delivery of therapeutics to the posterior of the eye while reducing procedure-related trauma. In some implementations, the method is extended to other dilatory and catheter-insertion applications elsewhere in the body. In brief, the disclosure discusses iteratively injecting liquid through a catheter to form a bubble near the distal tip of the catheter. The liquid is withdrawn to create a void, and the catheter is advanced into the void. The process is repeated until the distal tip of the catheter reaches the target location.

### CROSS-REFERENCE TO RELATED PATENT APPLICATIONS

This application claims priority from Provisional U.S. Patent Application 61/756,304, filed on Jan. 24, 2013, incorporated herein by reference in its entirety.

### BACKGROUND

The eye includes many layers of tissue surrounding a gelatinous substance called the vitreous. The white part of the eye seen from the exterior is called the sclera, and it is covered by a thin, transparent layer called the conjunctiva. The retina is the light-sensitive layer that enables vision, and it is in contact with the choroid, which contains a number of blood vessels.

While principles such as diffusion may be used for delivery of drugs to the posterior of the eye to treat some conditions, delivery of therapeutics requires a more invasive procedure. Typically, a catheter is inserted into an incision made through the sclera and advanced to target location. As with any invasive procedure, there are inherent risks. While the delivery catheter is relatively flexible, it is quite stiff along the insertion direction, which can lead to perforations of the retina. Large tissue distension caused by the procedure can be traumatic to the eye. Sometimes these retinal detachments and the resultant deleterious effects on vision may not occur immediately, making it more difficult to address the issue as well as to determine the specific origin and location.

### SUMMARY

According to one aspect of the disclosure, a method for inserting a catheter two layers of tissue. The method also includes iteratively, injecting, through a first lumen defined by the catheter, a liquid to form a bubble. The bubble has a first volume. Next, a portion of the first volume used to form the bubble is withdrawn through the catheter, and the catheter is advanced further between the first layer of tissue and the second layer of tissue.

In some implementations, advancing the distal tip of the catheter occurs concurrently with the withdrawing of the portion of the first volume. In certain implementations, the method further includes determining that the distal tip of the catheter is at a target location. In some implementations, a therapeutic agent is injected through the catheter, into the posterior of the eye. The therapeutic agent can be injected through a second lumen of the catheter.

In some implementations, the portion of the first volume is withdrawn through the catheter is substantially equal to the first volume of the liquid injected through the first lumen. In some implementations, the first volume is injected and withdrawn according to a sinusoidal flow pattern.

In certain implementations, the method also includes measuring a pressure near the distal tip of the catheter, and adjusting the first volume responsive to the measured pressure near the distal tip of the catheter. In some implementations, about 0.01 μL to about 10 μL of the liquid is injected to form the bubble. The diameter of the catheter is between about 250 μm and about 400 μm. In some implementations, the liquid is sodium hyaluronate. In some implementations, the first layer of tissue and the second layer of tissue are both layers of tissue of an eye.

According to another aspect of the disclosure, a system for inserting between two layers of tissue includes a catheter with a distal tip, a proximal end, and defining first lumen. The system also includes a first pump coupled to the proximal end of the catheter, and a controller coupled to the first pump. The controller is configured to cause the first pump to iteratively inject, through the first lumen a first volume of liquid to form a bubble near the distal tip of the catheter and withdraw a portion of the first volume through the catheter prior to or concurrently with each advancement of the catheter further between the two layers of tissue.

In some implementations, the system also includes a light source coupled to the proximal end of the catheter. The catheter includes a flow sensors, pressure sensors, and radio-opaque portions in some implementations. In some implementations, the tip of the catheter is beveled and includes a plurality of outlets. The diameter of the catheter is between about 250 μm and about 400 μm. In some implementations, the portion of the first volume withdrawn through the catheter is substantially equal to the first volume injected through the first lumen of the catheter.

### BRIEF DESCRIPTION OF THE DRAWINGS

The skilled artisan will understand that the figures, described herein, are for illustration purposes only. It is to be understood that in some instances various aspects of the described implementations may be shown exaggerated or enlarged to facilitate an understanding of the described implementations. In the drawings, like reference characters generally refer to like features, functionally similar and/or structurally similar elements throughout the various drawings. The drawings are not necessarily to scale, emphasis instead being placed upon illustrating the principles of the teachings. The drawings are not intended to limit the scope of the present teachings in any way. The system and method may be better understood from the following illustrative description with reference to the following drawings in which:

FIG. 1 illustrates an example system for delivering a therapeutic agent to the posterior of the eye.

FIGS. 2A-2D illustrate example catheter tip configurations that may be employed in the catheter of FIG. 1.

FIG. 3 illustrates a flow chart of an example method for inserting a catheter toward the posterior of the eye.

FIGS. 4A-4C illustrates a dilatory bubble separating two layers of tissue.

FIG. 5 shows an example of the relative timing of the steps in the method of FIG. 3.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

The method and system disclosed herein uses a pulsatile, dilatory bubble at the tip of a catheter to facilitate tissue separation and ease catheter insertion for, among other applications, delivery of therapeutics to the posterior of the eye, while reducing procedure-related trauma. In some implementations, the method is extended to other dilatory and catheter-insertion applications elsewhere in the body—for example, in small areas in which the larger resultant diameter of a balloon may not be usable. Throughout the disclosure, the delivery of a liquid toward the target destination through a catheter may be described as inject, dispense, or irrigate. Similarly, the removal of liquid may be described as withdraw, evacuate, or aspirate.

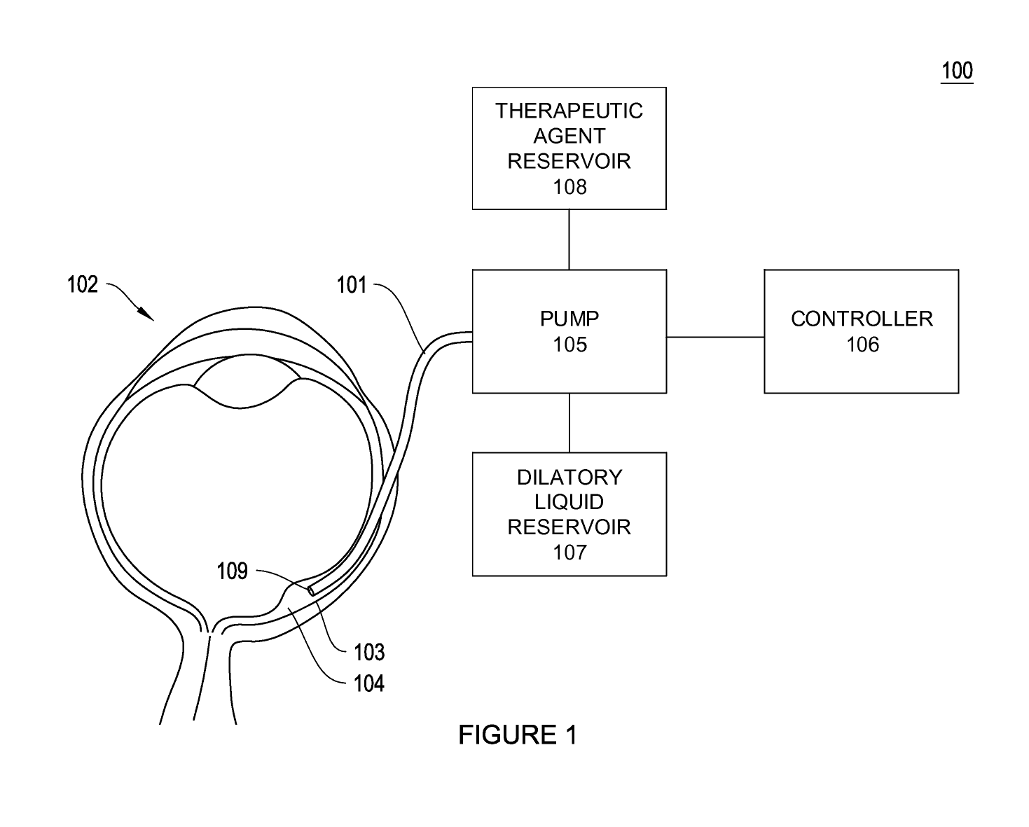

FIG. 1 illustrates a system 100 for delivering a therapeutic agent to the posterior of the eye 102. In general, the system 100 is used to advance a catheter 101 toward the posterior of the eye 102 and deliver the therapeutic agent once the tip 109 of the catheter 101 reaches a target destination. In some implementations, the catheter 101 is inserted between two layers of tissue, such as the choroid 103 and retina 104, along the periphery of the eye 102. The system 100 creates a dilatory bubble ahead of the catheter tip 109, reducing trauma as the catheter 101 is gradually advanced toward the posterior of the eye 102.

The system 100 includes a pump 105 coupled to the catheter 101. The pump 105 is controlled by a controller 106. The controller 106 actuates the pump 105 to flow a fluid into and out of the catheter 101. The system 100 also includes a dilatory liquid reservoir 107 and a therapeutic agent reservoir 108 from which the pump 105 draws fluid. In some implementations, the system 101 also includes a waste reservoir, used to collect waste fluid withdrawn from posterior of the eye 102 by the catheter 101.

The pump 105 can be any medical grade pump. As described below, the pump 105 is configured to generate a plurality of flow profiles, as controlled by the controller 106. In some implementations, the flow profile includes, but is not limited to, flow rate, flow direction, total volume injected (or withdrawn), flow duration, and flow waveform (e.g., square wave or sinusoidal wave). In some implementations, the pump 105 is a syringe coupled to a bi-directional syringe pump. The bi-directional syringe pump is controlled by the controller 106 to accomplish the below described inject/withdraw pulses. The syringe pump includes a motor-driven linear actuator that uses a helical/screw drive to convert the rotation of the motor into a linear displacement. The linear displacement depresses the plunger of a syringe and causes liquid to be dispensed. In implementations where the pump 105 is a syringe coupled to a bi-directional syringe pump, the dilatory liquid reservoir 107 and the therapeutic agent reservoir 108 are the barrels of the syringes coupled to the bi-directional syringe pump. For example, during the below described catheter advancement stage, a syringe filled with sodium hyaluronate (marketed as HEALON™ by Abbott Medical Optics, headquartered in Santa Anna, Calif.) is coupled to the bi-directional syringe pump. In this example, when the distal tip 109 of the catheter 101 has reached the target destination the sodium hyaluronate filled syringe is replaced with a syringe filled with a therapeutic agent. In some implementations, the syringe containing the therapeutic agent is coupled to the bi-directional syringe pump before the distal tip 109 of the catheter reaches the target destination to account for the dead volume of the catheter 101. For example, if x μL is ejected from the catheter 101 for every y linear millimeters traveled and the dead volume of the catheter 101 is V. Then the syringe containing the therapeutic agent may be replaced (Vy)/x mm away from the target such that the therapeutic agent is substantially at the tip 109 of the catheter 101 when the catheter 101 reaches the target destination.

In other implementations, the pump 105 is a peristaltic pump coupled to the catheter 101. The peristaltic pump includes a drive motor coupled to a pump head. As the motor rotates, the multiple rollers on the pump head impinge upon a flexible segment of tubing and at least partially occlude the tubing. The occlusion causes a localized increase in pressure that moves a fixed bolus of fluid through the tubing. Reversing the direction of the motor reverses the flow of liquid, and causes a withdrawal of fluid from the catheter 101. In other implementations, the pump 105 is a piezoelectrically-driven membrane at the proximal end of the catheter 101.

In some implementations, the dilatory liquid and the therapeutic liquids are different. In other implementations, the therapeutic agent itself may be used as the dilatory liquid. In some implementations, the injected fluid is any sterile liquid, such as saline. In some implementations, the liquid has a high relative viscosity to enable the dilatory bubble to separate layers of tissue. For example, the liquid may be sodium hyaluronate or related substances. Using the therapeutic agent along the catheter insertion path is an effective way to accomplish diffused therapeutic delivery. In some implementations, repeated changeover between dilatory and therapeutic liquids at key points along a catheter insertion path serves a similar purpose of distributed delivery while reducing the volumetric use of what may be an expensive therapeutic agent.

The therapeutic agents can include, but are not limited to, pharmacological agents, stem cells, cell-based therapeutics, protein/peptide-based therapeutics, genetic material, bacterial agents, viral vectors, whole blood, or blood components.

The controller 106 controls the pump 105. In some implementations, the controller 106 is a general purpose computing device. For example, the controller 106 can be a laptop, tablet computer, or smartphone. In other implementations, the controller 106 is a special purpose computer device and includes one or more processors and at least one computer readable medium, such as a hard drive, compact discs, or other storage device. Processor executable instructions are stored on the computer readable medium. When executed, the instructions cause the controller 106 to perform the functions and methods described herein. For example, the controller 106 controls the pump 105 to flow liquid from the dilatory liquid reservoir 107 into the catheter 101 at a predetermined rate.

In some implementations the catheter 101 includes one or more sensors, and the controller 106 receivers data from the sensors to set flow parameters, such as, but not limited to flow rate, flow direction, flow profile, pressure, or a combination thereof, responsive to the data received from the sensors.

To reduce ancillary tissue trauma, the bubble size and/or pressure may be monitored. Monitoring the size of the bubble may be accomplished via an imaging system such as ultrasound optical coherence tomography (OCT), and the pressure may be monitored via a pressure transducer fluidically coupled to the catheter 101. In addition to being used by the controller 106, the data may be transmitted to a display so that the user may modify the insertion and/or pulsatile profiles appropriately. In some implementations, the algorithm that controls the flow profile incorporates the flow parameters, catheter insertion input, and/or catheter pressure in conjunction with a computational/analytic fluidic model to provide similar pulsatile flow profiles under different conditions. In some implementations, the controller 106 tracks the location of the catheter tip 109 within the eye 102.

In some implementations, the controller 106 is pre-preprogrammed by a user to automatically inject/withdraw liquid responsive to the advancement of the catheter 101. In some implementations, the pump 105 is controlled by a push button, a foot pedal, voice command, or other user input.

The system also includes a catheter 101. The catheter can be any medical grade catheter. In some implementations, the catheter in is any type of conduit or channel such as, but not limited to, a cannula, a micro-cannula, a microbore, a tube, or endoscope. In some implementations, the catheter 101 is a needle. The diameter of the catheter is between about 100 μm and about 2 mm, between about 100 μm and about 250 μm, between about 250 μm and about 1 mm, between about 250 μm and about 500 μm, between about 250 μm and about 400 μm, or between about 250 μm and about 350 μm. The catheter includes at least one internal lumen. In some implementations, the catheter 101 includes a plurality of lumens. For example, the catheter 101 can include a first lumen for the delivery of the dilatory liquid and a second lumen for the therapeutic agent. As described below, the distal tip 109 of the catheter 101 can include different tip configurations, such as, but not limited to, different tip shapes or multiple outputs.

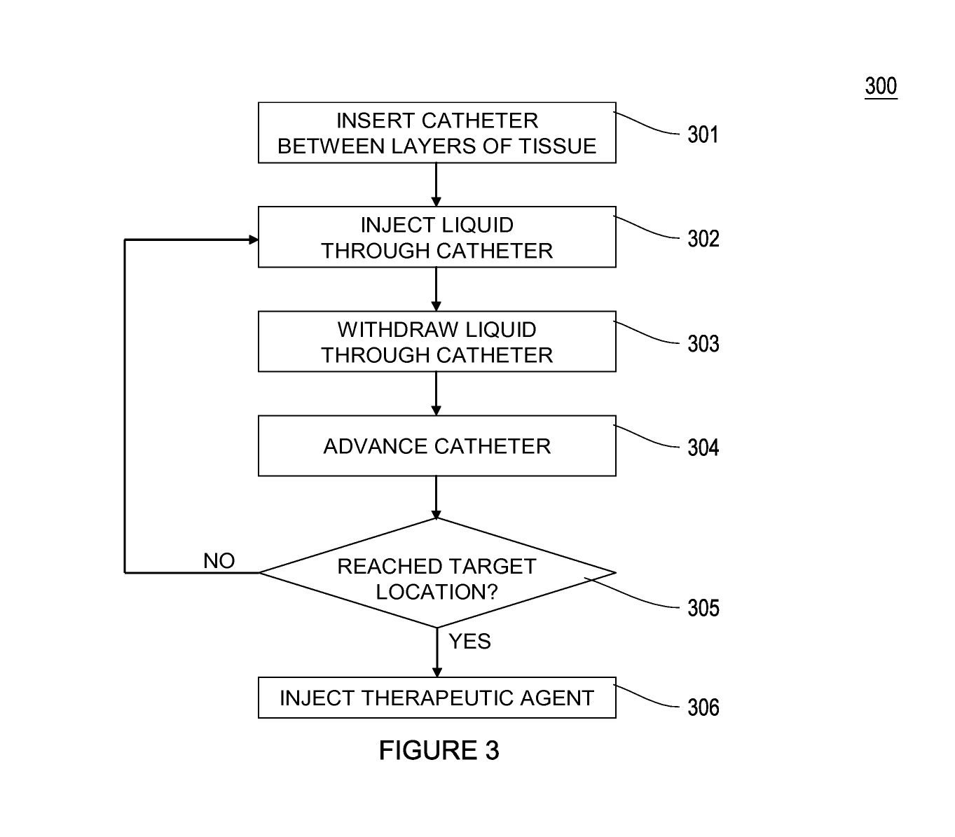

In some implementations, the catheter 101 is a component of or fed through a handpiece. The handpiece can include micro-manipulators that advance the catheter 101 toward the posterior of the eye 102. In some implementations, the advancement of the catheter 101 by the micro-manipulators of the handpiece is controlled by the controller 106. For example, a physician may push a button on the handpiece, indicating that he wishes the catheter to be advanced a predetermined distance toward the target location. The controller 106 may then initiate the catheter advancement method described in relation to FIG. 3.

In some implementations, the catheter 101 includes depth markings along the length of the catheter 101. In some implementations, detecting the arrival of the catheter 101 at the target location is achieved by a user's visual observation of a given depth marking on the catheter 101. The depth marking indicates the correct insertion depth has been achieved. In other implementations, the tip 109 is tracked with optical tracking by an operative-field camera or fundoscope that detects and tracks the motion of insertion depth-markings on the catheter or an optical encoder mounted near or on the catheter. Other encoder types commonly used in the field may also be applied to track the catheter insertion depth.

In some implementations, the body of the catheter 101 includes a fiber optic cable or the wall of the catheter 101 is configured to transmit light along the length of the catheter 101. In some implementations, the catheter 101 includes a radio opaque material that enables the catheter 101 to be visualized in a radiograph. In some implementations, the catheter 101 includes sensors, such as, but not limited to, temperature, pressure, flow sensors, or any combination thereof.

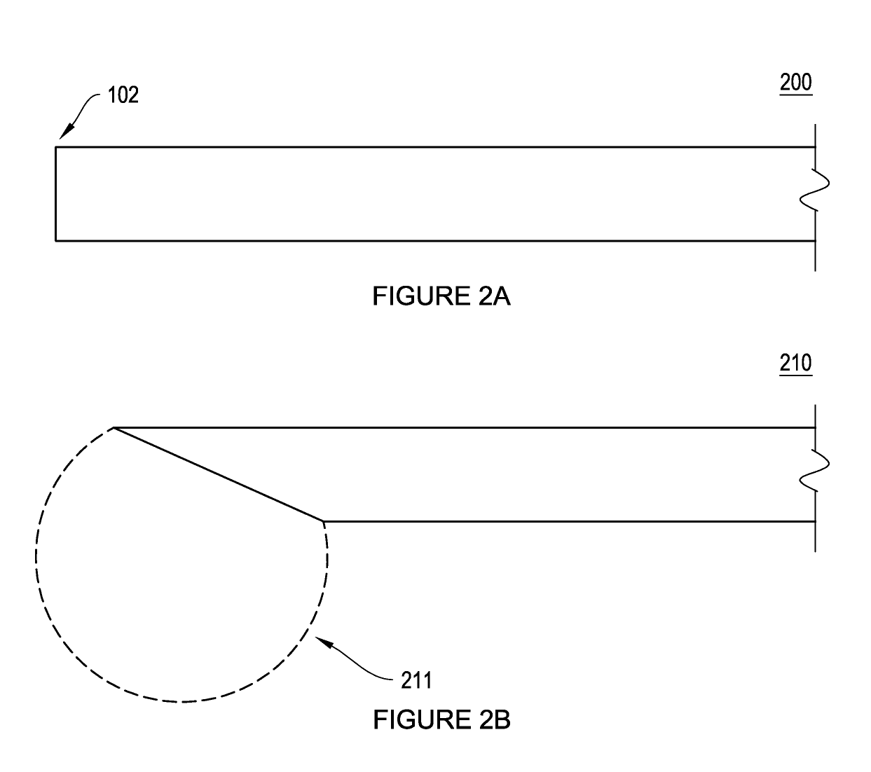

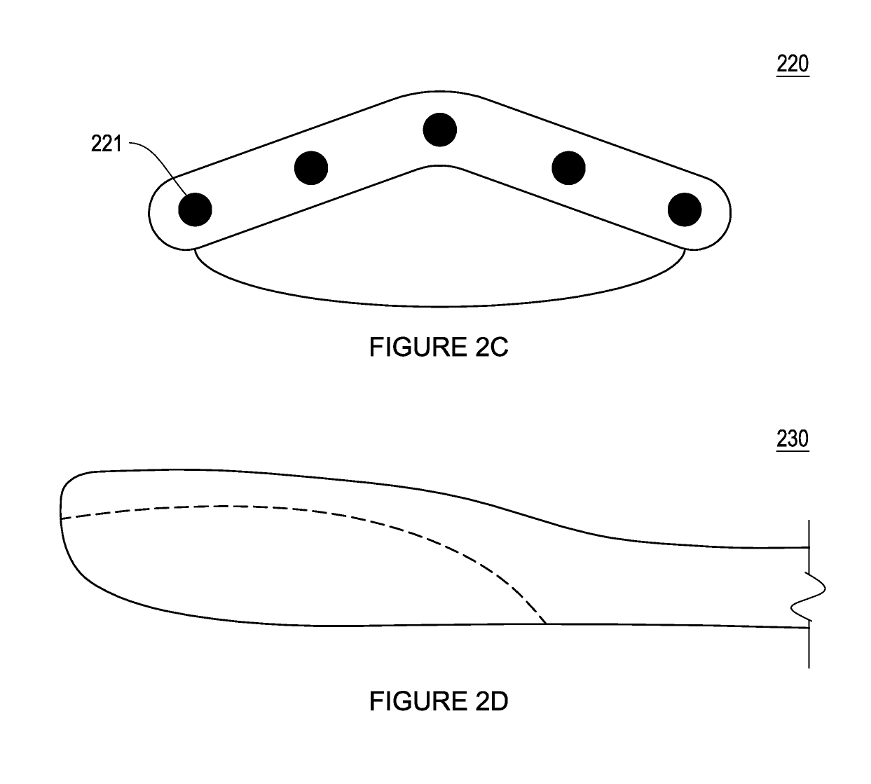

FIGS. 2A-2D illustrate example catheter tip configurations. FIG. 2A illustrates a catheter tip 200 with a blunt tip configuration. In some implementations, a blunt tip creates a substantially symmetrical dilatory bubble. A symmetrical bubble projects substantially an equal distance in one direction above and below the central axis of the catheter. In some implementations, the edges 201 of the tip 200 are rounded to reduce the likelihood of the tip causing trauma during the insertion process. In other implementations, the tip 200 tapers towards outlet of the tip 200.

FIG. 2B illustrates a tip 210 with a bevel. In some implementations, the bevel ranges from about 89° to about 10°, from about 60° to about 10°, from about 35° to about 10°, or from about 20° to about 10°. In some implementations, the beveled tip 210 is used to create an asymmetrical dilatory bubble 211. In general, asymmetrical dilatory bubbles 211 projects a greater distance above or below the central axis of the catheter. For example, the tip 210 creates the bubble 211, which projects a greater distance below the central axis of the catheter compared to above the central axis of the catheter. An asymmetrical dilatory bubble 211 may be used if, when separating the tissue layers, the physician wishes to place more force on one tissue layer than the other tissue layer.

FIG. 2C illustrates a front view of a rounded scoop catheter tip configuration 220, and FIG. 2D illustrates a side profile of the catheter tip configuration 220. FIG. 2C illustrates that the tip configuration 220 includes a plurality outputs 221 for the release of liquid. In some implementations, the tip of the catheter is configured to reduce the possible trauma it may cause to tissue during the advancement of the catheter. In some implementations, the tip of the catheter is configured for the dissection of tissue.

FIG. 3 illustrates a flow chart of an example method 300 for inserting a catheter toward the posterior of the eye. The method 300 includes inserting the catheter between two layers of tissue (step 301). Next, a liquid is injected through the catheter (step 302). The liquid is then withdrawn through the catheter (step 303) and the catheter is advanced toward the posterior of the eye (step 304). A determination is made whether the tip of the catheter has reached the target location (step 305). If the target location has not been reached, the steps of injecting the fluid, withdrawing the fluid, and advancing the catheter are repeated. If the target location has been reached a therapeutic agent is injected through the catheter (step 306).

As set forth the above, the method 300 begins with the insertion of the catheter between two layers of tissue (step 301). In some implementations, the two layers of tissue are the choroid and the retina of the eye. In some implementations, access to the two layers of tissue is provided through a small incision in the sclera of the eye. The system described herein may also be used in other medical procedures where a catheter or endoscope is advanced between layers of tissue. For example, the system described herein may be used to separate adipose tissue from muscle tissue during plastic surgery, urinary surgery, pediatric surgery, or other procedures.

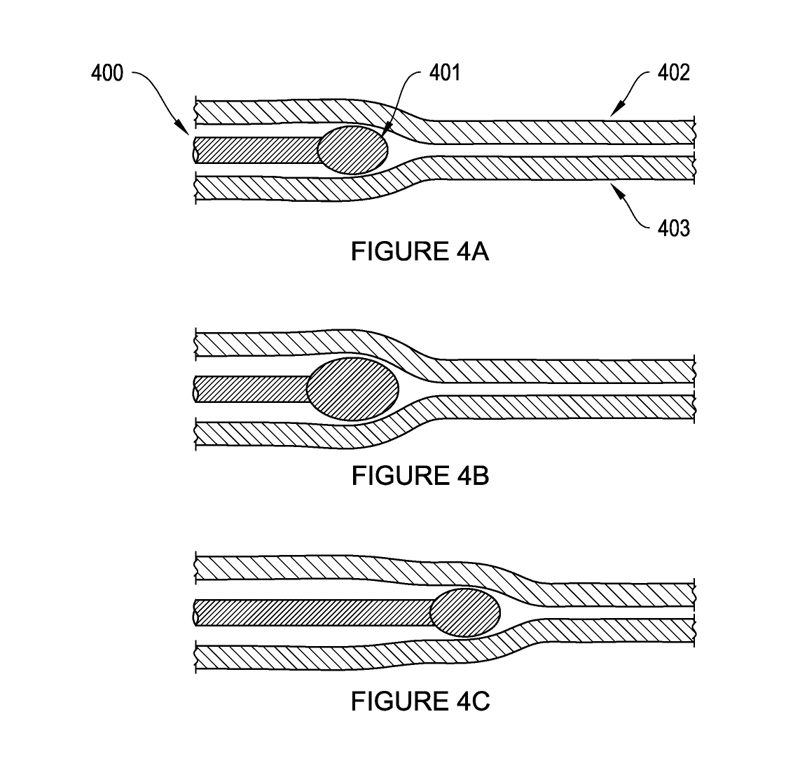

Next, and referring to FIG. 4A, a liquid is injected through the catheter to separate the layer of tissue (step 302). FIGS. 4A-4C illustrates a dilatory bubble separating two layers of tissue. In FIG. 4A, a fluid is dispensed form a catheter 400 to form a dilatory bubble 401. In some implementations, as the dilatory bubble 401 increases in size the resultant increase in pressure separates a first tissue layer 402 from a second tissue layer 403. For example, in an opthalmologic application, a 30-gauge catheter (approximately 300 μm diameter) may be used to create a dilatory bubble between the layers of tissue. In some implementations, the volume of the dilatory bubble is between about 5 μL and about 150 μL, between about 5 μL and about 100 μL, between about 5 μL and about 50 μL, between about 5 μL and about 25 μL, or between about 0.01 μL and about 5 μL. The separation of the first tissue layer 402 and the second tissue layer 403 creates a void, into which the catheter 400 can be advanced. FIG. 4B illustrates the bubble at its maximum size.

Next, and also referring to FIG. 4C, the liquid is withdrawn through the catheter (step 303). In FIG. 4C, a fraction of the volume of the dilatory bubble is withdrawn through the catheter 400 to reduce the total volume of fluid introduced to the site. In some implementations, substantially the same amount of liquid that is injected through the catheter is also withdrawn back through the catheter. In some implementations, the catheter has a second lumen used for the withdrawal of the fluid through the catheter. For example, the catheter may include a first lumen for injecting the fluid and a second lumen for withdrawing the fluid such that “fresh” fluid is always injected between the layers of tissue. In some implementations, a higher injection volume compared to the withdrawn volume helps keep the tissue slightly separated from the catheter and prevents bodily fluid from being drawn into the catheter. In some implementations, a small expanding bubble of liquid at the tip of the catheter helps to gently separate the tissue layers without delivering a high volume of liquid that would expand the separation to an area far wider than that of the catheter. In an opthalmological application, it is advantageous when separating the choroid and retina to reduce the size of the retinal detachment. In some implementations, a judicious introduction of liquid reduces the risk of elevated intraocular pressure (IOP). In other implementations, such as a plastic surgery application, reducing the size of the affected area reduces negative cosmetic effects.

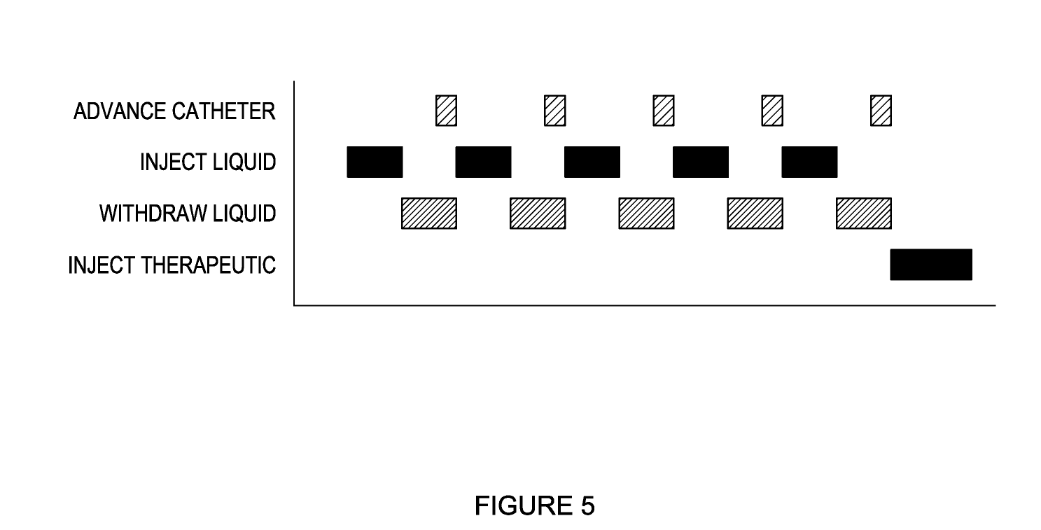

Referring back to FIG. 3 and also to FIG. 5, once the liquid is at least partially withdrawn, the catheter is advanced toward the posterior of the eye (step 304). In some implementations, the catheter is advanced concurrently with the withdrawal of the dilatory bubble. In other implementations, the catheter is advanced after the completion of the withdrawal step described above. In some implementations, the advancement distance is substantially equivalent to the diameter of the dilatory bubble (i.e., the catheter is advanced to the perimeter of the void created by the dilatory bubble. In some implementations, the catheter is advanced between about 2 μm and about 1.5 mm, between about 2 μm and about 1 mm, between about 5 μm and about 500 μm, between about 5 μm and about 250 μm, between about 5 μm and about 125 μm, between about 5 μm and about 50 μm, or between about 2 μm and about 25 μm. In some implementations, the catheter is advanced a length equivalent to about 3 times the diameter of the catheter. FIG. 5 is a diagram illustrating an example timing of the above described steps 302-304. FIG. 5 illustrates each injection and withdrawal as continuous, but in some implementations, each sequence is discontinuous. For example, the injection may include injecting a plurality of smaller boluses that are cumulatively the above described dilatory bubble.

As illustrated in FIG. 5, the example injection and withdrawal cycles take substantially the same amount of time. In other implementations, the injection or withdrawal steps take different lengths of time. In some implementations, the ratio of the injection cycle to the withdrawal cycle is 70:30. In some implementations, the length of time of the injection and withdrawal steps is different, but the system injects and withdraws liquid the same amount of liquid. For example, the injection step may be shorter but at a higher relative flow rate compared to the withdrawal step. In some implementation, there is a predetermined rest period between subsequent injection and withdrawal sequences. As illustrated in FIG. 5 and described above, in some implementations, the catheter is advanced at the end of a withdrawal sequence. In other implementations, the catheter is advanced concurrently with the injection of the liquid, concurrently with the withdrawal of the fluid, or between withdrawal and injection sequences.

Referring again back to FIG. 3, after the catheter is advanced, a determination is made whether the catheter tip has reached the target location (step 305). As described above, the placement of the catheter tip can be determined through visual inspection or by reading markings along the length of the catheter. Once the catheter tip reaches the target destination, a therapeutic agent is injected through the catheter (step 306). As described above, the therapeutic agent may be the dilatory liquid used in the advancement of the catheter toward the posterior of the eye. In other implementations, the therapeutic agent is a second liquid that is flowed through the catheter once the distal tip of the catheter reaches the target location. In some implementations in which the catheter includes only a single lumen, the dilatory fluid is fully withdrawn from the catheter before the therapeutic is introduced. In some implementations, after the dilatory liquid has been fully withdrawn, an appropriate dosage of the therapeutic is introduced into the catheter. A sterile fluid, such as the dilatory fluid, is then introduced behind the therapeutic to force substantially the entire dose into the desired location without utilizing excess volume of the therapeutic.

In some implementations, the pulsatile liquid previously described may be used to provide less-traumatic insertion of a catheter or other conduit for the purpose of withdrawing some bodily substance. Thus, upon reaching a given target location, a substance is withdrawn from the body.

In another implementation, the pulsatile liquid previously described may be used to provide less-traumatic insertion of a catheter or other conduit for the purpose of gaining access to a location for viewing, imaging, performing some diagnostic procedure, or performing some therapeutic procedure. Thus, upon reaching a given target location, viewing, imaging, some diagnostic procedure, and/or some therapeutic procedure may occur. In some of these implementations, an additional instrument (or instrument connection) may be inserted through the lumen of the catheter, such as an optical fiber, conductive wires, or other device.

The invention may be embodied in other specific forms without departing from the spirit or essential characteristics thereof. The forgoing implementations are therefore to be considered in all respects illustrative, rather than limiting of the invention.

What is claimed:

1. A method for inserting a catheter between two layers of tissue, the method including:
inserting a distal tip of a catheter between a first layer of tissue and a second layer of tissue;
iteratively, until a target destination is reached;
injecting, through a first lumen defined by the catheter, a liquid to form a bubble having a first volume at the distal tip of the catheter between the first layer of tissue and the second layer of tissue;
withdrawing, through the catheter, a portion of the first volume used to form the bubble; and
advancing the distal tip of the catheter further between the first layer of tissue and the second layer of tissue.

2. The method of claim 1, wherein advancing the distal tip of the catheter occurs concurrently with the withdrawing of the portion of the first volume.

3. The method of 1, further comprising determining the distal tip of the catheter is at a target location.

4. The method of claim 1, further comprising injecting a therapeutic agent through the catheter.

5. The method of claim 4, wherein injecting the therapeutic agent further comprises injecting the therapeutic agent through a second lumen of the catheter.

6. The method of claim 1, wherein the portion of the first volume withdrawn through the catheter is substantially equal to the first volume of the liquid injected through the first lumen.

7. The method of claim 1, where the first volume is injected and withdrawn according to a sinusoidal flow pattern.

8. The method of claim 1, further comprising:
measuring a pressure near the distal tip of the catheter; and
adjusting the first volume responsive to the measured pressure near the distal tip of the catheter.

9. The method of claim 1, wherein injecting the liquid further comprises injecting between about 0.01 μL and about 10 μL.

10. The method of claim 1, wherein a diameter of the catheter is between about 250 μm and about 400 μm.

11. The method of claim 1, wherein the liquid is sodium hyaluronate.

12. The method of claim 1, wherein the first layer of tissue and the second layer of tissue are both layers of tissue of an eye.

13. A device for inserting a catheter between two layers of tissue, the device comprising:
a catheter with a distal tip, a proximal end, and defining first lumen;
a first pump coupled to the proximal end of the catheter; and
a controller coupled to the first pump, the controller configured to cause the first pump to iteratively inject, through the first lumen a first volume of liquid to form a bubble near the distal tip of the catheter and withdraw a portion of the first volume through the catheter prior to or concurrently with each advancement of the catheter further between the two layers of tissue.

14. The device of claims 13, further comprising a light source coupled to the proximal end of the catheter.

15. The device of claim 13, wherein the portion of the first volume withdrawn through the catheter is substantially equal to the first volume.

16. The device of claim 13, further comprising a sensor toward the distal tip of the catheter.

17. The device of claim 16, wherein the sensor is one of a pressure sensor and a flow sensor.

18. The device of claim 13, the catheter further comprising a radio opaque portion towards the distal tip of the catheter.

19. The device of claim 13, wherein the distal tip has a plurality of fluid outlets.

20. The device of claim 13, wherein the distal tip is beveled.

21. The device of claim 13, wherein a diameter of the catheter is between about 250 μm and about 400 μm.
